SEQUENCE LISTING (10 sequences)

SEQ ID NO: 1 - protein, 976 aa, Homo sapiens
MELQAARACFALLWGCALAAAAAAQGKEVVLLDFAAAGGELGWLTHPYGKGWDLMQNIMNDMPIYMYSVCNVMSGDQDNWLRTNWVYRGEAERIFIELKFTVRDCNSFPGGASSCKETFNLYYAESDLDYGTNFQKRLFTKIDTIAPDEITVSSDFEARHVKLNVEERSVGPLTRKGFYLAFQDIGACVALLSVRVYYKKCPELLQGLAHFPETIAGSDAPSLATVAGTCVDHAVVPPGGEEPRMHCAVDGEWLVPIGQCLCQAGYEKVEDACQACSPGFFKFEASESPCLECPEHTLPSPEGATSCECEEGFFRAPQDPASMPCTRPPSAPHYLTAVGMGAKVELRWTPPQDSGGREDIVYSVTCEQCWPESGECGPCEASVRYSEPPHGLTRTSVTVSDLEPHMNYTFTVEARNGVSGLVTSRSFRTASVSINQTEPPKVRLEGRSTTSLSVSWSIPPPQQSRVWKYEVTYRKKGDSNSYNVRRTEGFSVTLDDLAPDTTYLVQVQALTQEGQGAGSKVHEFQTLSPEGSGNLAVIGGVAVGVVLLLVLAGVGFFIHRRRKNQRARQSPEDVYFSKSEQLKPLKTYVDPHTYEDPNQAVLKFTTEIHPSCVTRQKVIGAGEFGEVYKGMLKTSSGKKEVPVAIKTLKAGYTEKQRVDFLGEAGIMGQFSHHNIIRLEGVISKYKPMMIITEYMENGALDKFLREKDGEFSVLQLVGMLRGIAAGMKYLANMNYVHRDLAARNILVNSNLVCKVSDFGLSRVLEDDPEATYTTSGGKIPIRWTAPEAISYRKFTSASDVWSFGIVMWEVMTYGERPYWELSNHEVMKAINDGFRLPTPMDCPSAIYQLMMQCWQQERARRPKFADIVSILDKLIRAPDSLKTLADFDPRVSIRLPSTSGSEGVPFRTVSEWLESIKMQQYTEHFMAAGYTAIEKVVQMTNDDIKRIGVRLPGHQKRIAYSLLGLKDQVNTVGIPI

SEQ ID NO: 2 - protein, 9 aa, Artificial Sequence
  Synthetic Peptide
IVGAETFYV

SEQ ID NO: 3 - protein, 10 aa, Artificial Sequence
  Synthetic Peptide
RPHERNGFTV

SEQ ID NO: 4 - protein, 9 aa, Artificial Sequence
  Synthetic Peptide
IMNDMPIYM

SEQ ID NO: 5 - protein, 9 aa, Artificial Sequence
  Synthetic Peptide
DMPIYMYSV

SEQ ID NO: 6 - protein, 9 aa, Artificial Sequence
  Synthetic Peptide
VLLLVLAGV

SEQ ID NO: 7 - protein, 9 aa, Artificial Sequence
  Synthetic Peptide
VLAGVGFFI

SEQ ID NO: 8 - protein, 9 aa, Artificial Sequence
  Synthetic Peptide
TLADFDPRV

SEQ ID NO: 9 - protein, 9 aa, Artificial Sequence
  Synthetic Peptide
YMPIYMYSV

SEQ ID NO: 10 - protein, 13 aa, Artificial Sequence
  Synthetic Peptide
TPPAYRPPNAPIL

(19) United States
(12) Patent Application Publication
(10) Pub. No.: US 2006/0034856 A1
(43) Pub. Date: Feb. 16, 2006

(54) Epha2 antigen t epitopes

(76) Inventors: Kostas Kosmatopoulos, Paris (FR); Pedro Alves, Meudon (FR)

Correspondence Address: OBLON, SPIVAK, MCCLELLAND, MAIER & NEUSTADT, P.C., 1940 DUKE STREET, ALEXANDRIA, VA, 22314, US

(21) Appl. No.: 10/511,273

(22) PCT Filed: Apr. 23, 2003
(86) PCT No.: PCT/FR03/01280

§ 371 Date: Jun. 27, 2005

(30) Foreign Application Priority Data
Apr. 23, 2002 (FR) 02/05048

(51) Int. Cl. A61K 39/00; C07K 14/74

(52) U.S. Cl. 424/185.1; 530/350

(57) ABSTRACT

The invention concerns peptides constituting T epitopes of EphA antigen, exhibited by MHC I. Said peptides are useful in particular in antitumoral immunotherapy.

The present invention relates to peptides derived from the EphA2 protein and to their use in antitumor immunotherapy.

Peptide immunization or immunotherapy is a therapeutic approach which is currently the subject of a great deal of interest in the context of the prevention or treatment of cancer. The principle thereof is based on immunization with peptides which reproduce T epitopes of tumor antigens recognized by cytotoxic T lymphocytes (CTLs), which play a major role in the elimination of cancer cells expressing these antigens at their surface.

It will be recalled that CTLs do not recognize whole protein antigens, but peptide fragments thereof, presented by major histocompatibility complex (MHC) molecules expressed at the surface of various cells. It is these peptide fragments which constitute the T epitopes.

The presentation of these peptides is the result of a complex process, called “antigen processing”, which involves 3 main steps:
cytosolic degradation of the antigens by a multienzyme complex called proteasome;
translocation of the peptides derived from this degradation into the endoplasmic reticulum (ER) by TAP transporters;
association of these peptides with the MHC so as to form stable peptide/MHC complexes which will be exported to the cell surface.

The presentation of T epitopes at the cell surface depends in particular on the stability of the antigenic protein in the cytosol, on the sites and on the frequency of the cleavages carried out by the proteasome, on the efficiency of translocation into the ER by the TAP transporters, and on the ability of the peptides to bind to the MHC molecules and to form stable peptide/MHC complexes.

The epitopes presented by the major histocompatibility complex class I (MHC I) generally have 8 to 11 amino acids, and are recognized by CD8+ T cells, which represent the major component of the cytotoxic response. The epitopes presented by the major histocompatibility complex class II (MHC II) generally have 13 to 18 amino acids and are recognized by CD4+ T cells.

The identification of these epitopes, and in particular (given the essential role of the CD8+ response in cytotoxicity) of those presented by MHC I, therefore constitutes an essential step in the development of antitumor immunotherapy compositions.

Many tumor antigens capable of inducing a CTL response are known at the current time. Some of the T epitopes of these antigens have been identified, and the effectiveness of vaccines based on peptides which reproduce these T epitopes has been shown in many cases. However, the expression of the majority of these antigens is restricted to certain histological types of tumors, which limits their clinical use. It is therefore desirable to identify other tumor antigens expressed by a large number of tumors of varied origin, and which are also capable of inducing an antitumor cytotoxic immune response.

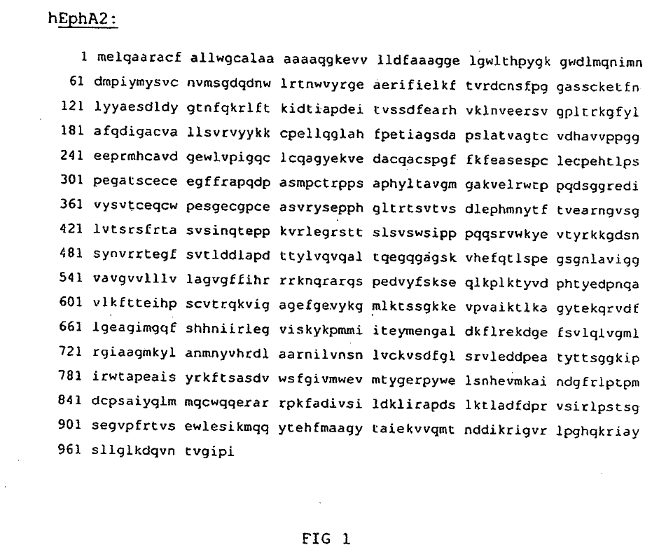

The EphA2 receptor, previously called ECK (Lindberg and Hunter, Molec. Cell. Biol. 10, 6316-6324, 1990), is a membrane receptor which has tyrosine kinase activity. The sequence of the human EphA2 receptor is represented in FIG. 1 (SEQ ID NO: 1). This receptor comprises an extracellular domain of 534 amino acids, a transmembrane domain of 24 amino acids and a cytoplasmic domain of 418 amino acids which contains tyrosine kinase domain. This receptor is overexpressed in several types of cancer, such as colon cancer, breast cancer, prostate cancer, lung cancer, stomach cancer, esophageal cancer and metastatic melanoma, but is not overexpressed in non-cancerous lesions in these same tissues (Rosenberg et al. Am. J. Physiol. 273, 824, 1997; Zelinski et al. Cancer Res. 61, 2301, 2001; Nemoto et al. Pathobiology 65, 195, 1997; Easty et al. Int. J. Cancer 60, 129, 1995; Walker Daniel et al. Prostate 41, 275, 1999). It has been observed that the overexpression of EphA2 is linked to malignant transformation and facilitates the metastatic progression of tumors. In addition, EphA2 plays an important role in tumor neovascularization (Ogawa et al. Oncogene 19, 6043, 2000).

Due to its overexpression in many types of tumors, and to its involvement in malignant transformation and in tumor angiogenesis, it has been proposed to use EphA2 as a target for antitumor treatments. Thus, PCT application WO 01/121172 proposes the use of antibodies directed against B epitopes carried by the extracellular domain of the EphA2 receptor for passive antitumor immunotherapy.

However, it was not known, until now, whether EphA2 could be effectively processed so as to generate T epitopes capable of inducing an antitumor cytotoxic response. A fortiori, no T epitope of this antigen had been identified.

The inventors have now identified, in EphA2, immunogenic peptides presented by MHC I which induce cytotoxic T lymphocytes capable of lysing tumor cells expressing EphA2.

Consequently, a subject of the present invention is an immunogenic peptide constituting a T epitope presented by MHC I, characterized in that it consists of a fragment of 8 to 11 consecutive amino acids of the EphA2 antigen.

In the context of the disclosure of the present invention, the term “immunogenic peptide” is intended to mean a peptide capable of inducing a specific CTL response against the EphA2 antigen.

Peptides in accordance with the invention can be obtained from the EphA2 antigen in various ways. For example, it is known that peptides capable of forming a complex with a given MHC I allele have in common the presence, at certain positions, of specific amino acid residues called “anchor residues”. Specific anchor motifs involving amino acids called “primary anchor residues” have thus been defined for the various MHC I alleles. It has also been shown that residues located outside the primary anchor motifs (secondary anchor residues) can exert a favorable or unfavorable effect on the affinity of the peptide for MHC.

The choice of the peptide sequences capable of constituting epitopes presented by a given MHC I allele can be made, conventionally, by analyzing the peptide sequence of the EphA2 antigen in order to select the peptides which have all or part of the primary anchor motif corresponding to this allele. Various databases which list the known anchor motifs are available: by way of examples, mention will be made of the SYFPEITHI base (http://www.uni-tuebingen.de/uni/kxi/; Rammensee et al., Immunogenetics, 50, 213-219, 1999), or the BIMAS base (http://bimas.dcrt.nih.gov/molbio/hla bind; Parker et al., J. immunol. 152, 163, 1994).

Generally, the binding affinity of the peptides thus identified for the allele of the concerned will then be determined, as will the stability of the peptide/MHC I molecule complex. In fact, non-immunogenic peptides most commonly exhibit weak affinity for MHC I molecules, and/or form with them a relatively unstable complex. Methods for determining the affinity of the peptide for the MHC I molecule and the stability of the complex formed are known per se. Mention will be made, for example, of that described by Firat et al. (Eur. J. Immunol., 29, 3112, 1999).

The affinity of a peptide for an MHC I molecule is most commonly defined with respect to that of a reference peptide (for example IVGAETFYV (SEQ ID NO: 2) for HLA-A*0201 or RPHERNGFTV (SEQ ID NO: 3) for HLA-B*0702), in the form of relative affinity. The relative affinity is defined as the ratio of the concentration of the peptide tested to the concentration of the reference peptide which allows the formation, under the same conditions, of the same amount of peptide/MHC I molecule complex. The higher the relative affinity, the lower the binding affinity of the peptide for the MHC I molecule.

The stability of the peptide/MHC I molecule complex is often defined by the DC₅₀, which represents the time required for the dissociation of 50% of the complexes formed.

For example, in the case of potentially immunogenic peptides presented by HLA-A*0201, the relative affinity is generally less than 5 and the DC₅₀ greater than 2 hours.

The immunogenicity of the potentially immunogenic peptides thus detected can be verified, for example by means of conventional methods for determining the ability of this peptide to generate, in vivo, ex vivo or in vitro, a specific CTL response with respect to target cells loaded with this peptide, or expressing the EphA2 antigen from which it is derived.

The peptides which have a weak affinity for the MHC I molecule concerned, and/or which form with the latter a relatively unstable complex, generally have a weak immunogenicity. However, these peptides may be of therapeutic interest insofar as it appears that low-affinity epitopes do not contribute, or only slightly contribute, to the establishing of tolerance phenomena, which constitute one of the main pitfalls of antitumor immunization.

In this case, it is possible to prepare variant peptides which have greater immunogenicity, by substitution of one or more of the amino acids of the native peptide with one or more amino acids favorable to the affinity for the MHC I molecule concerned and/or to the stability of the peptide/MHC I molecule complex.

These variant peptides are also part of the subject of the present invention.

Amino acids that are favorable to the affinity for a given MHC I molecule and/or to the stability of the peptide/MHC I molecule complex may, for example, consist of anchor residues, and in particular the secondary anchor residues, known for the MHC I molecule concerned. These anchor residues can be readily identified by consulting the available databases, such as those mentioned above.

By way of example of substitution making it possible to increase the immunogenicity of a peptide presented by an MHC I molecule, and in particular by HLA-A*0201, mention will be made of the substitution of the N-terminal amino acid of said peptide with a tyrosine, as described in PCT application WO 02/08716.

The affinity of a variant peptide for the MHC I molecule concerned, and also its immunogenicity, can then be verified as indicated above for the native peptides.

By way of nonlimiting example of implementation of the present invention, the inventors have identified five peptides, hereinafter referred to as p58, p61, p546, p550 and p883, presented by HLA-A*0201.

The sequences (1-letter code) of these peptides are as follows:

| | | |
|---|---|---|
| p58: | IMNDMPIYM; | (SEQ ID NO: 4) |
| p61: | DMPIYMYSV; | (SEQ ID NO: 5) |
| p546: | VLLLVLAGV; | (SEQ ID NO: 6) |
| p550: | VLAGVGFFI; | (SEQ ID NO: 7) |
| p883: | TLADFDPRV. | (SEQ ID NO: 8) |

The inventors have also obtained, from the p61 peptide, which exhibits only weak affinity for HLA-A*0201 and weak immunogenicity, an immunogenic peptide, hereinafter referred to as p61Y, of sequence YMPIYMYSV (SEQ ID NO: 9), resulting from the substitution of the N-terminal residue of p61 with a tyrosine residue.

These peptides are capable of inducing a specific CTL response with respect to HLA-A*0201 cells expressing EphA2. They induce in particular a cytotoxic response with respect to HLA-A*0201 tumor cells derived from tumors of varied types.

A subject of the present invention is also compositions comprising at least one immunogenic peptide in accordance with the invention, or a nucleic acid molecule encoding said peptide.

They may be multiepitope compositions capable of generating a polyspecific CTL response, and which, with the same, also comprise one or more other immunogenic epitope(s). These other epitopes may be derived from EphA2 or from one or more other antigens.

These multiepitope compositions in accordance with the invention may comprise, so that they can be widely used on a population whose individuals carry different HLA alleles, epitopes presented by various MHC I molecules. They may also comprise, in addition, at least one epitope presented by an MHC II molecule and capable of inducing a T-helper response.

According to a preferred embodiment of a composition in accordance with the invention, it comprises at least one chimeric polypeptide comprising one or more copies of an immunogenic peptide in accordance with the invention. In the case of a multiepitope composition, said chimeric polypeptide also comprises one or more copies of at least one other immunogenic epitope.

Such a chimeric polypeptide can be readily obtained by methods known per se, and in particular by conventional recombinant DNA techniques.

A subject of the present invention is also the nucleic acid molecules encoding an immunogenic peptide or a chimeric polypeptide in accordance with the invention.

A subject of the present invention is also the use of an immunogenic peptide epitope, of a composition or of a nucleic acid molecule in accordance with the invention, for obtaining a medicinal product, and in particular a medicinal product intended for antitumor immunotherapy, and in particular for the treatment of tumors expressing EphA2.

This encompasses a large variety of tumors, among which mention will in particular be made of colon tumors, breast tumors, prostate tumors, lung tumors, stomach tumors, kidney tumors and esophageal tumors.

The p58, p61, p546, p550, p883 and p61Y peptides can in particular be used for obtaining medicinal products intended for the treatment of HLA-A*0201 patients.

The present invention also encompasses the medicinal products comprising, as active principle, at least one immunogenic peptide, one composition or one nucleic acid molecule in accordance with the invention.

According to a preferred embodiment of the present invention, said medicinal products are vaccines.

Medicinal products in accordance with the invention can also comprise the usual excipients, and also adjuvants conventionally used in immunotherapy and which make it possible, for example, to promote the administration of the active principle, to stabilize it, to increase its immunogenicity, etc.

The present invention will be understood more thoroughly from the further description which follows, which refers to nonlimiting examples illustrating the induction of an antitumor cytotoxic response by peptides in accordance with the invention derived from the EphA2 antigen.

### EXAMPLE 1

### Identification of EphA2 Epitopes Presented by the HLA-8*0201 Molecule

The amino acid sequence of the EphA2 protein was analyzed by means of the BIMAS program (Parker et al., J. Immunol. 152, 163, 1994), in order to identify peptides potentially capable of binding to HLA-A*0201. Among the potential epitopes identified, the following five peptides:

| | | |
|---|---|---|
| p58: | IMNDMPIYM; | (SEQ ID NO: 4) |
| p61: | DMPIYMYSV; | (SEQ ID NO: 5) |
| p546: | VLLLVLAGV; | (SEQ ID NO: 6) |
| p550: | VLAGVGFFI; | (SEQ ID NO: 7) |
| p883: | TLADFDPRV; | (SEQ ID NO: 8) |

were selected.

The peptides corresponding to these sequences were synthesized by SYNT:EM (Nimes, France). The purity (>85%) is monitored by reverse-phase high performance liquid chromatography. The peptides are lyophilized and then dissolved in DMSO at 10 mg/ml and stored at −80° C.

The immunogenicity of these peptides was evaluated by measuring their affinity for HLA-A*0201. This is defined by two parameters: the relative affinity (RA) which reflects the ability of the peptides to bind to HLA-A*0201, and the rate of dissociation of the HLA-A*0201/peptide complexes (DC₅₀), which reflects their stability. The high-affinity peptides (RA<5 and DC₅₀>2 hrs) are potentially immunogenic, unlike the low-affinity peptides (RA>5 and DC₅₀<2 hrs).

Relative Affinity:

Human T2 cells (Firat et al., Eur. J. Immunol., 29, 3112, 1999) (3×10⁵ cells/ml), which are deficient in TAP transporters, are incubated at 37° C. for 16 hours with various concentrations (100 μM, 10 μM, 1 μM, 0.1 μM) of each test peptide, in serum-free RPMI 1640 medium supplemented with 100 ng/ml of human β2-microglobulin. Next, they are washed twice and labeled with the monoclonal antibody BB7.2 (Parham et al., Hum. Immunol., 3, 4, 277-299, 1981) which is specific for the HLA-A*0201 molecule, and then with a goat anti-mouse Ig antibody coupled to fluorescein isothiocyanate (FITC).

These cells are then analyzed by flow cytometry. For each concentration of peptide, the fluorescence specific for HLA-A*0201 is calculated as a percentage of the fluorescence obtained with 100 μM of a reference peptide (HIVpol 589; IVGAETFYV; SEQ ID NO: 2). The relative affinity (RA) is defined as the ratio of the concentration of each peptide that induces 20% of the fluorescence obtained with 100 μM of the reference peptide, to the concentration of the reference peptide that induces 20% of the fluorescence obtained with 100 μM of said reference peptide. The lower the relative affinity, the stronger the binding of the peptide to HLA-A*0201. The mean RA for each peptide is determined from at least three independent experiments. In all the experiments, 20% of the maximum fluorescence was obtained for 1 to 3 μM of the reference peptide.

Stability:

T2 cells (10⁶/ml) are incubated overnight at 37° C. with 100 μM of each test peptide, in serum-free RPMI 1640 medium supplemented with 100 ng/ml of human β2-microglobulin. Next, they are washed four times in order to remove the free peptides, incubated with BREFELDIN A (Sigma; 10 μg/ml) for one hour in order to prevent the expression at their surface of newly synthesized HLA-A*0201 molecules, washed, and incubated at 37° C. for 0, 2, 4, 6 or 8 hours in the presence of BREFELDIN A (0.5 μg/ml). For each incubation time, the cells are then labeled, as indicated above, with the BB7.2 antibody and analyzed by flow cytometry in order to evaluate the amount of peptide/HLA-A*0201 complex present at their surface. This amount is evaluated by means of the formula: (mean fluorescence of the T2 cells preincubated with the peptide−mean fluorescence of the T2 cells treated under similar conditions in the absence of peptide). The DC₅₀ (dissociation complex: DC) is defined as being the time (in hours) required for the loss of 50% of the HLA-A*0201/peptide complexes which are stabilized at t=0.

The results of these experiments are given in Table 1 below.

TABLE 1

| Peptide | Sequence | RA | DC₅₀ |
|---|---|---|---|
| p58 | IMNDMPIYM | 1 | 4 |
| p61 | DMPIYMYSV | >10 | ND |
| P61Y | YMPIYMYSV | 1.5 | ND |
| p546 | VLLLVLAGV | 1.4 | 4-6 |
| p550 | VLAGVGFFI | 1 | 4-6 |
| p883 | TLADFDPRV | 2.2 | 2-4 |

ND: not determined

These results show that the p58, p546, p550 and p883 peptides have a considerable binding affinity (RA of 1 to 2.2). On the other hand, the p61 peptide has a weak affinity for HLA-A*0201 (RA>10) and should therefore not be immunogenic. In order to improve the affinity of this peptide for HLA-A*0201, the inventors substituted the aspartic acid at position 1 with a tyrosine residue. The variant peptide p61Y obtained exhibits a considerable binding affinity (RA=1.5) that is clearly greater than that of the peptide from which it is derived.

The results also show that the p58, p546, p550 and p883 peptides form stable complexes with the HLA-A*0201 molecules (DC₅₀>2 h for each of them).

### EXAMPLE 2

### Immunogenicity of the P58, p61Y, p546, p550 and p883 Peptides

Induction of Specific CTLs by Immunization with the Peptides

The immunogenicity of the p58, p61Y, p546, p550 and p883 peptides was evaluated, by generation of CTLs, on HHD transgenic mice (Pascolo et al., J. Exp. Med., 185, 2043, 1997). These mice are β2m−/−, D^{b}−/− and express an HLA-A*0201 single chain made up of the α1 and α2 domains of HLA-A*0201 and of the α3 and intracellular domains of D^{b}, linked via its N-terminal end to the C-terminal end of human β2-microglobulin by means of a 15 amino acid peptide.

The HHD mice receive a subcutaneous injection, at the base of the tail, with 100 μg of each test peptide emulsified in incomplete Freund's adjuvant, in the presence of 140 μg of a T-helper epitope derived from the HBV “core” antigen (128-140, sequence TPPAYRPPNAPIL, SEQ ID NO: 10).

After 11 days, spleen cells taken from the mice (5×10⁷ cells in 10 ml) are stimulated in vitro with the test peptide (10 μM). On the 6th day of the culture, the populations which respond are tested in order to determine a specific cytotoxicity. The cells which respond are restimulated in vitro at one-week intervals with 2×10⁷ irradiated (3000 rads) HHD spleen cells and 1 to 0.1 μM of peptide in the presence of 50 IU/ml of recombinant IL2 (Proleukin, Chiron Corp.)

Cytotoxicity assays are carried out 6 days after the final stimulation.

RMAS-HHD cells are used as targets to study the cytotoxicity. These cells are obtained by transfection of murine RMAS cells with the HHD construct as described by Pascolo et al. (J. Exp. Med., 185, 2043, 1997).

These target cells are labeled with 100 μCi of ⁵¹Cr for 90 minutes, and then washed three times and plated out in round-bottomed 96-well plates (3×10³ cells/well in 100 μl of RPMI 1640+3% of fetal calf serum). They are loaded with 1 μM of the test peptide, or of an irrelevant control peptide, at 37° C. for 90 minutes.

Next, 100 μl of the effector cells (effector cells/target cell ratio=40/1) are added to the wells and the plates are incubated at 37° C. for 4 hours. After incubation, 100 μl of supernatant are collected and the radioactivity is measured in a γ-counter.

The percentage specific lysis is calculated by means of the formula [(experimental ⁵¹Cr release−spontaneous ⁵¹Cr release)/(maximum ⁵¹Cr release−spontaneous ⁵¹Cr release)]×100. In all the experiments, the spontaneous release is less than 20% of the maximum release induced with 3N HCl.

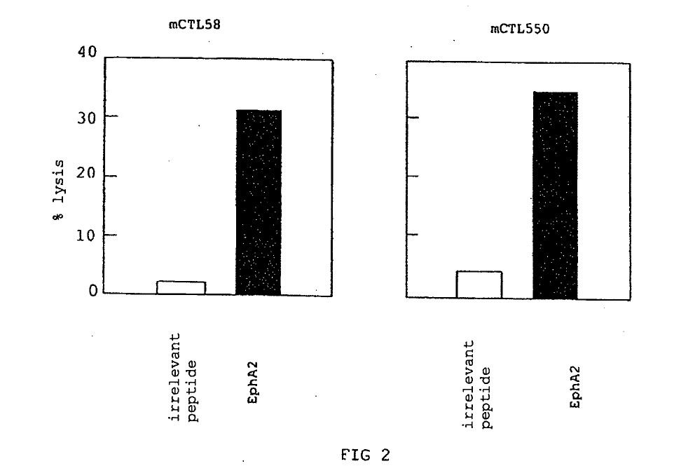

The results of these experiments for the p58 and p550 peptides are given in FIG. 2.
□: irrelevant peptide;
▪: EphA2 peptide.

These results show that immunization with the p58 or p550 peptide generates CTLs which kill the RMAS-HHD targets loaded with this same peptide, but not the cells loaded with the irrelevant peptide. Equivalent results were obtained with the p61Y, p546 and p883 peptides.

CTL lines, respectively called mCTL58, mCTL61Y, mCTL546, mCTL550 and mCTL883, were established from the spleen cells of HDD mice immunized with the p58, p61Y, p546, p550 or p883 peptide, by repeated stimulation in vitro with the decreasing concentrations (10 μM-1 μM) of the same peptide.

The avidity of these lines for their inducer peptide was determined by measuring, as described above, their cytotoxicity with respect to RMAS-HHD target cells loaded with increasing concentrations (1 pM to 10 μM) of the peptide concerned.

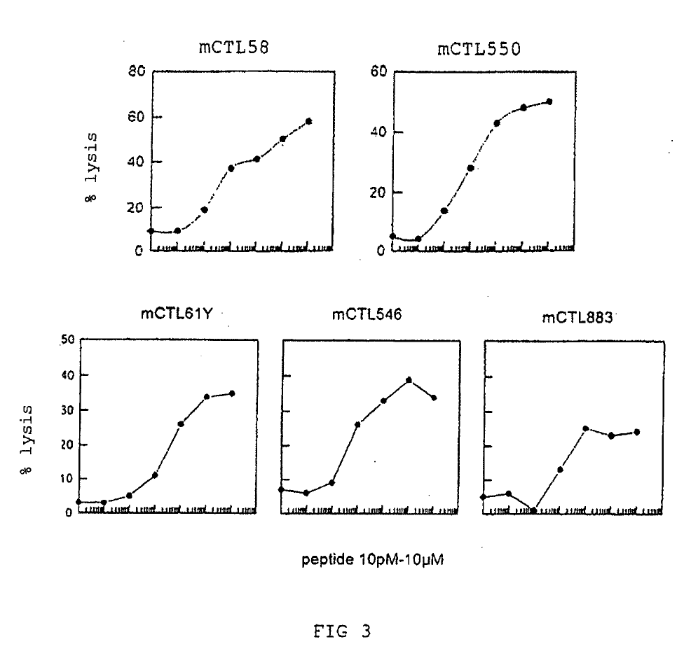

The results are given in FIG. 3.

These results show that the mCTL58, mCTL61Y, mCTL546, mCTL550 and mCTL883 lines have a relatively high avidity. 50% of maximum lysis is obtained for peptide concentrations which range from 3 nM in the case of mCTL546 to 40 nM in the case of mCTL61Y.

### EXAMPLE 3

### Recognition of the Naturally Processed Epitopes of the EphA2 Antigen by CTLs Induced by the p58 or p550 Peptides

To test whether the p58 and p550 peptides constitute naturally processed epitopes of the EphA2 antigen, the response of the cells of the mCTL58 and mCTL550 lines to cells expressing this antigen was evaluated in two different ways.

1) Stimulation with Transfected COS-7 Cells Expressing EphA2

The cells of the mCTL58 and mCTL550 lines are stimulated with monkey COS-7 cells cotransfected with the HHD construct (Pascolo et al., mentioned above) and a plasmid containing the EphA2 cDNA. COS-7 cells transfected either with the HHD construct alone or with the plasmid containing the EphA2 cDNA alone are used as negative controls.

The CTL stimulation is evaluated by measuring their TNF-α secretion. The COS-7 cells transfected with the HHD construct and loaded with the p58 or p550 peptide are used as a positive control.

Four days after transfection, the COS-7 cells are brought into contact with the mCTL58 and mCTL550 cells in a proportion of 5×10⁴ CTLs per 3×10⁴ COS-7 cells, in RPMI 1640 in the presence of 10% SVF.

After incubation for 6 hours, the supernatant is removed (50 μl) and brought into contact with WEHI164 clone 13 mouse fibrosarcoma cells (3×10⁴ per well), which are characterized by a high sensitivity to TNF-α-induced apoptosis. In order to quantify the TNF content in the culture supernatant, a standard range of TNF-α (concentrations of 0 to 10⁴ pg/ml) is used in parallel. After incubation at 37° C. for 16 hours, the viability of the WEHI-164 clone 13 cells is determined by means of an MTT colorimetric assay (SIGMA) (Espevik and Nissen Meyer, J. Immunol. Methods., 95, 99, 1986).

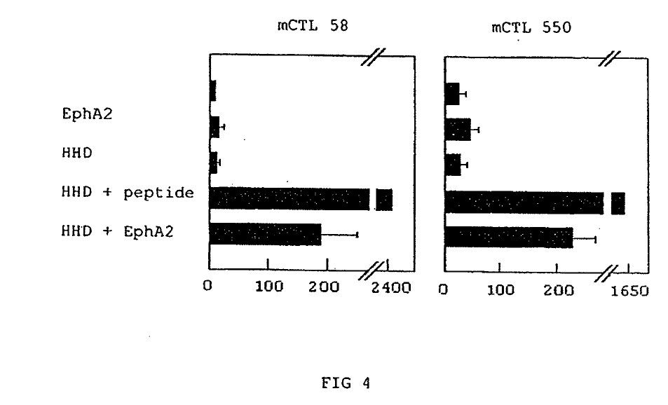

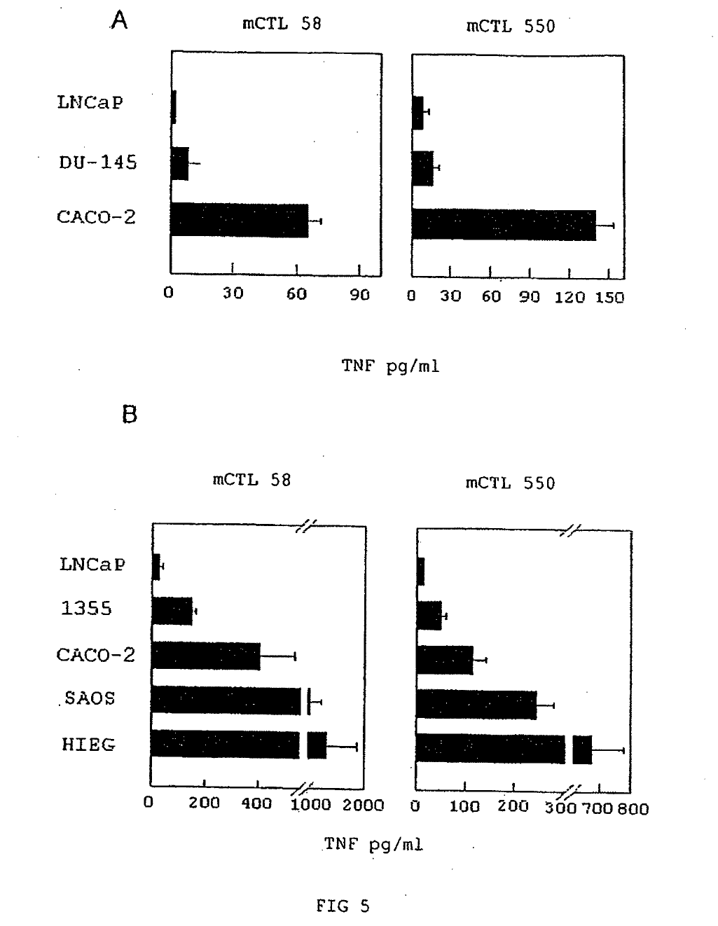

The results are given in FIG. 4.
-: untransfected COS-7 cells;
EphA2: COS-7 cells transfected with the EphA2 cDNA alone;
HHD: COS-7 cells transfected with the HHD construct alone;
HHD+peptide: COS-7 cells transfected with the HHD construct and loaded with the p58 or p550 peptide;
HHD+EphA2: COS-7 cells transfected with the HHD construct and the EphA2 cDNA.

These results show that the mCTL58 and mCTL550 lines respond to stimulation with the COS cells coexpressing HHD and EphA2.

On the other hand, no response is observed to the COS cells transfected separately with the HHD construct or with the EphA2 cDNA.

2) Stimulation with HLA-A*0201 Human Tumor Cells Expressing EphA2

The following HLA-A*0201 tumor lines were used: SAOS (sarcoma), 1355 (lung cancer), Caco-2 (colon cancer), HIEG (renal carcinoma), LNCaP (prostate cancer). The DU145 line (prostate cancer), which does not express HLA-A*0201, was also used as a negative control.

Among these lines, DU145 and Caco-2 are known to express EphA2, and LNCaP is known not to express EphA2.

The expression of EphA2 in the other tumor lines was evaluated by Western blotting. The level of EphA2 expression in all the lines used is summarized in Table II below.

TABLE II

| | HLA-A*0201 | |
|---|---|---|
| Cell line | expression | EphA2 expression |
| SAOS | + | + |
| 1355 | + | + |
| Caco-2 | + | + |
| HIEG | + | + |
| LNCaP | + | − |
| DU145 | − | + |

+: strong expression
−: no expression.

The mCTL58 and mCTL550 lines were stimulated with the SAOS, 1355, Caco-2, HIEG, LNCaP and DU145 tumor lines mentioned above. The mCTL61Y, mCTL546 and mCTL 883 lines were stimulated with the LNCaP, DU145 and Caco-2 tumor lines mentioned above. The stimulation is evaluated by the detection of TNF-α secretion as described above.

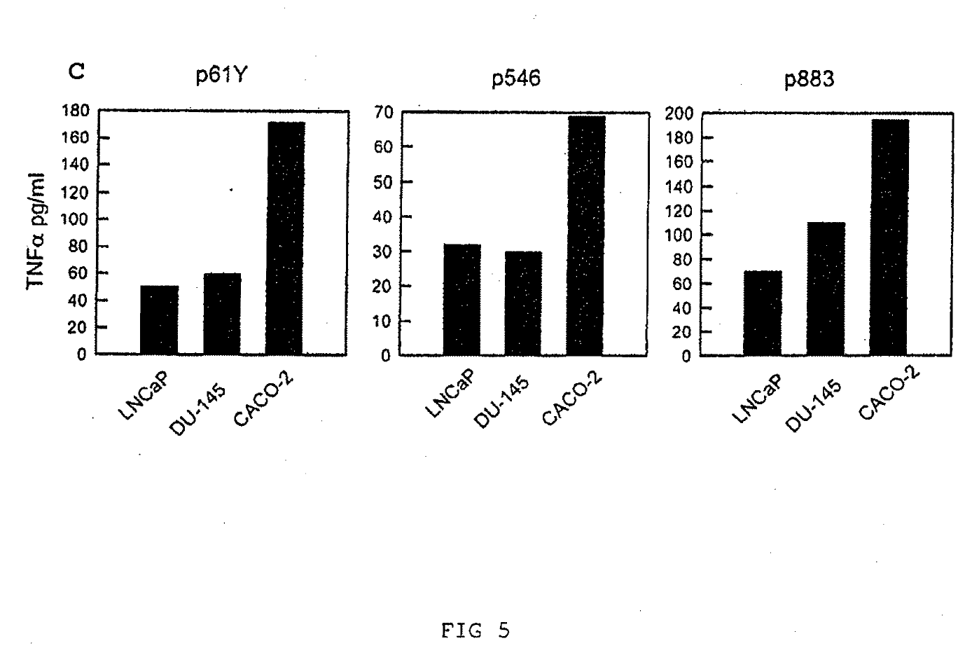

The results are given in FIGS. 5A, 5B and 5C.

FIG. 5A shows that the mCTL58 and mCTL550 cells respond to the stimulation with the Caco-2 cells, which express HLA-A*0201 and EphA2, but respond neither to the DU145 cells, which do not express HLA-A*0201, nor to the LNCaP cells, which do not express EphA2.

FIG. 5B shows that the mCTL58 and mCTL550 cells respond to the stimulation with the HIEG, Caco-2, 1355 and SAOS cells, which express large amounts of EphA2, but do not respond to the LNCaP cells, which do not express EphA2.

FIG. 5C shows that the mCTL61Y, mCTL546 and mCTL883 cells respond to the stimulation of the Caco-2 cells, which express large amounts of EphA2, but do not respond to the LNCaP or DU145 cells, which do not express EphA2 and HLA-A*0201, respectively.

The results of the above experiments show that the CTLs induced by p58, p61Y, p546, p550 or p883 recognize naturally processed epitopes of the EphA2 antigen.

### EXAMPLE 4

### Induction of Human CTLs Specific for the p58 or p550 Peptides

The ability of p58 and p550 to induce CTLs in vitro from peripheral blood mononuclear cells (PMBCs) from normal donors was tested as follows.

The PBMCs are obtained, from blood samples taken by leukocytapheresis on normal donors, after centrifugation at 2000 rpm for 20 min on a Ficoll/Hypaque (Amersham) gradient. After 3 washes in 0.9% NaCl, 10⁷ PBMCs are resuspended in each of the wells of a 6-well culture plate, in 3 ml of complete medium (RPMI 1640 supplemented with 10% of heat-inactivated human AB serum), and incubated at 37° C. for 2 hours. After incubation, the cells which have not adhered are removed and the cells which have adhered are differentiated into dendritic cells by adding to each of the wells 3 ml of complete medium containing 50 ng/ml of GM-CSF (R & D Systems), and 1000 IU/ml of IL-4 (R & D Systems). After 7 days of culture, the dendritic cells are collected and loaded with the p58 or p550 peptide by incubation for 4 hours at 20° C. with 40 μg/ml of peptide in the presence of 3 μg/ml of β2-microglobulin, and are then irradiated at 4200 rads; they are subsequently washed in order to remove the free peptide. CD8+ cells are isolated from the non-adherent cells by means of microbeads coupled to an anti-CD8 antibody (Miltenyi Biotec).

0.5×10⁶ CD8+ cells are stimulated by coculture in a 48-well plate with 2.5×10⁴ dendritic cells loaded with the p58 or p550 peptide, in complete medium supplemented with 10 ng/ml of IL-7, in a final volume of 500 μl/well. On the day after the placing of the cells in culture, 10 ng/ml of human IL-10 (R & D Systems) are added to each of the wells; on the second day, 30 IU/ml of human IL-2 are added to each of the wells. Oh the seventh and the fourteenth day after the first stimulation, the CD8+ cells are restimulated with the adherent cells loaded with 10 μg/ml of peptide in the presence of 3 μg/ml of β2-microglobulin, and irradiated. IL-10 (10 ng/ml) and IL-2 (30 IU/ml) are added 24 hours and 48 hours, respectively, after restimulation. Seven days after the second restimulation, the response of these cells to T2 cells loaded with p58 or P550 or with an irrelevant peptide, or to Caco-2 HLA-A*0201 tumor cells (expressing EphA2 and HLA-A*0201), LNCaP HLA-A*0201 tumor cells (expressing HLA-A*0201 but not expressing EphA2) and DU145 tumor cells (expressing EphA2 but not expressing HLA-A*0201), is evaluated by assaying the intracellular IFNγ production.

The hCTL58 or hCTL550 cells are incubated with the loaded T2 cells, or with the cells of the tumor line tested, in the presence of 20 μg/ml of Brefeldin-A (Sigma). After 6 hours, they are washed, labeled with an anti-CD8 antibody conjugated to r-phycoerythrin (Caltag Laboratories) in PBS for 25 min at 4° C., washed and fixed with 4% paraformaldehyde. They are then permeabilized with saponin (Sigma) at 0.2% in PBS, and labeled with an anti-IFNγ monoclonal antibody conjugated to allophycocyanin (Pharmingen).

The cells are then analyzed by flow cytometry (FACSCalibur™ (Becton Dickinson) and the CellQuest™ program).

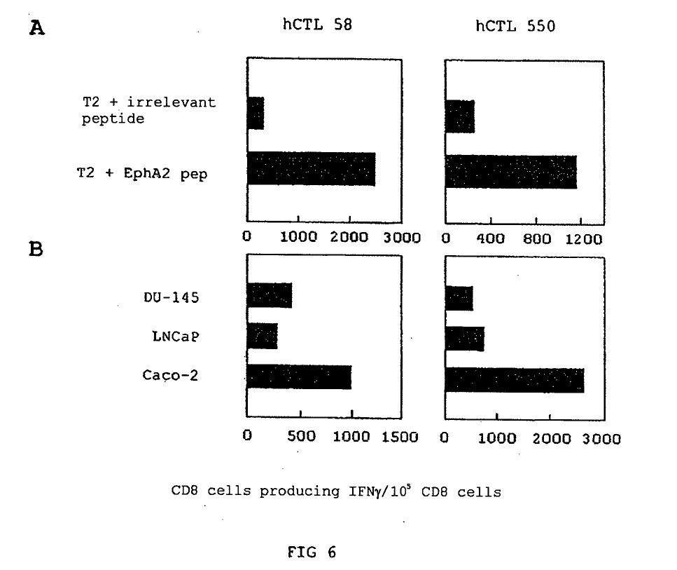

The results (expressed as number of CD8+ cells producing IFNγ per 10⁵ CD8+ cells, are given in FIGS. 6A and 6B.

FIG. 6A shows that the human CTLs obtained from CD8+ cells stimulated, respectively, with the p58 peptide (hCTL58) or the p550 peptide (hCTL550) are activated by the T2 cells loaded with the corresponding peptide, and that no activation by the T2 cells loaded with the irrelevant peptide is observed.

FIG. 6B shows a response of the hCTL58 and hCTL550 CTLs with respect to the Caco-2 tumor line (EphA2⁺, HLA-A*0201⁺), but not with respect to the LNCaP (EphA2⁻, HLA-A*0201⁺) and DU145 (EphA2⁺, HLA-A*0201⁻) lines.

These results demonstrate that the p58 or p550 peptides induce human CTLs capable of recognizing HLA-A*0201+tumor cells expressing EphA2.

1. An immunogenic peptide constituting a T epitope presented by MHC I, characterized in that it consists of a fragment of 8 to 11 consecutive amino acids of the EphA2 antigen.

2. The immunogenic peptide as claimed in claim 1, characterized in that it is chosen from:
the peptide of sequence IMNDMPIYM (SEQ ID NO: 4);
the peptide of sequence VLLLVLAGV (SEQ ID NO: 6);
the peptide of sequence VLAGVGFFI (SEQ ID NO: 7);
the peptide of sequence TLADFDPRV (SEQ ID NO: 8).

3. An immunogenic peptide constituting a T epitope presented by MHC I, characterized in that it is derived from a peptide consisting of a fragment of 8 to 11 consecutive amino acids of the EphA2 antigen, by substitution of at least one amino acid of said peptide with an amino acid which increases the affinity of said peptide for an MHC I allele.

4. The peptide as claimed in claim 3, characterized in that it is derived from a peptide consisting of a fragment of 8 to 11 consecutive amino acids of the EphA2 antigen, by substitution of the N-terminal amino acid of said peptide with a tyrosine residue.

5. The peptide as claimed in claim 4, characterized in that it is defined by the sequence YMPIYMYSV (SEQ ID NO: 9).

6. A polynucleotide encoding a peptide as claimed in any one of claims 1 to 5.

7. A composition comprising at least one peptide as claimed in any one of claims 1 to 6 or a polynucleotide as claimed in claim 5.

8. The composition as claimed in claim 7, characterized in that it is a multiepitope composition also comprising one or more other immunogenic peptide(s) or one or more polynucleotide(s) encoding said peptide(s).

9. The composition as claimed in claim 7, characterized in that it is a chimeric polypeptide comprising at least one copy of a peptide as claimed in any one of claims 1 to 5 and/or at least one copy of another immunogenic peptide, or a polynucleotide encoding said chimeric polypeptide.

10. The use of a peptide as claimed in any one of claims 1 to 5, of a polynucleotide as claimed in claim 6 or of a composition as claimed in any one of claims 7 to 9, for obtaining a medicinal product.

11. The use as claimed in claim 10, characterized in that said medicinal product is intended for antitumor immunotherapy.

12. The use as claimed in claim 11, characterized in that said medicinal product is intended for the immunotherapy of tumors expressing the EphA2 antigen.

13. The use as claimed in any one of claims 10 to 12, characterized in that said medicinal product is intended for the treatment of HLA-A*0201 patients.
